SEQUENCE LISTING (8 sequences)

SEQ ID NO: 1 - DNA, 18 bp, Artificial Sequence
  NOD2 forward primer
gccacggtgaaagcgaat

SEQ ID NO: 2 - DNA, 21 bp, Artificial Sequence
  NOD2 reverse primer
ggaagcgagactgagcagaca

SEQ ID NO: 3 - DNA, 21 bp, Artificial Sequence
  IFN-B forward primer
gattcatctagcactggctgg

SEQ ID NO: 4 - DNA, 20 bp, Artificial Sequence
  IFN-B reverse primer
cttcaggtaatgcagaatcc

SEQ ID NO: 5 - DNA, 24 bp, Artificial Sequence
  IL-8 forward primer
tgcagctctgtgtgaaggtgcagt

SEQ ID NO: 6 - DNA, 25 bp, Artificial Sequence
  IL-8 reverse primer
cagtgtggtccactctcaatcactc

SEQ ID NO: 7 - DNA, 20 bp, Artificial Sequence
  GAPDH forward primer
ttggtatcgtggaaggactc

SEQ ID NO: 8 - DNA, 20 bp, Artificial Sequence
  GAPDH reverse primer
acagtcttctgggtggcagt

(19) United States
(12) Patent Application Publication
(10) Pub. No.: US 2017/0021014 A1
(43) Pub. Date: Jan. 26, 2017

(54) VACCINE ADJUVANTS FOR CYTOMEGALOVIRUS PREVENTION AND TREATMENT

(71) Applicant: THE JOHNS HOPKINS UNIVERSITY, Baltimore, MD (US)

(72) Inventor: Ravit Boger, Baltimore, MD (US)

(21) Appl. No.: 15/215,711

(22) Filed: Jul. 21, 2016

Related U.S. Application Data
(60) Provisional application No. 62/194,908, filed on Jul. 21, 2015.

(51) Int. Cl. A61K 39/39; A61K 39/12

(52) U.S. Cl. CPC A61K 39/39; A61K 39/12; C12N 2710/00034; A61K 2039/5254

(57) ABSTRACT

The present invention relates to the field of virology. More specifically, the present invention provides methods and compositions useful for treating human cytomegalovirus using bacterial cell wall components MDP and tri-DAP as vaccine adjuvants. In specific embodiments, the present invention provides a pharmaceutical composition comprising a human cytomegalovirus vaccine and a NOD1 activator and/or a NOD2 activator. In particular embodiments, the NOD2 activator is muramyl dipeptide (MDP) or a derivative thereof. In certain embodiments, the NOD1 activator is L-Ala-γ-D-Glu-meso-diamino-pimelic acid (tri-DAP) or a derivative thereof.

### CROSS-REFERENCE TO RELATED APPLICATIONS

This application claims the benefit of U.S. Provisional Application No. 62/194,908, filed Jul. 21, 2015, which is incorporated herein by reference in its entirety.

### FIELD OF THE INVENTION

The present invention relates to the field of virology. More specifically, the present invention provides methods and compositions useful for prevention and treatment of human cytomegalovirus (CMV) using bacterial cell wall components MDP and tri-DAP as vaccine adjuvants.

### INCORPORATION-BY-REFERENCE OF MATERIAL SUBMITTED ELECTRONICALLY

This application contains a sequence listing. It has been submitted electronically via EFS-Web as an ASCII text file entitled “P13558-01_ST25.txt.” The sequence listing is 1,750 bytes in size, and was created on Jul. 21, 2015. It is hereby incorporated by reference in its entirety.

### BACKGROUND OF THE INVENTION

Infection with human CMV (CMV), a member of the herpes virus family, is common in humans. Seroprevalence rates increase with age, reaching 80-90% in individuals older than 80 years. The virus establishes lifelong persistent (latent) infection, usually asymptomatic.

CMV causes significant morbidity and mortality in congenitally-infected children and immunocompromised hosts. Approximately 40,000 newborns in the US (1% of live-born) are infected with CMV every year; some will be severely sick at birth and others will present later with hearing loss, usually around the time they enter elementary school. The importance of CMV as the leading infectious cause of mental retardation and deafness in children has been emphasized by its categorization as a level I vaccine candidate by the Institute of Medicine.

CMV disease is also a major cause of morbidity and mortality in bone marrow and solid organ transplant recipients. Prior to the availability of prophylactic antiviral therapy, 75% of transplant recipients experienced CMV infection or disseminated disease. In solid organ transplant recipients the highest risk occurs when the donor is CMV-positive and the recipient is CMV-negative, but infection is also common when both donor and recipient are CMV-positive.

Infection with CMV also contributes to the natural history of AIDS. CMV-seropositive HIV-infected patients progress 2.5-fold more rapidly to AIDS and death than those who are CMV-seronegative. Despite highly-active antiretroviral therapy (HAART), HIV-infected patients remain at risk for CMV disease. In 10-20% of patients treated with HAART the CD4 T-lymphocyte cell counts remain low, and a large number of patients do not receive HAART because of cost, noncompliance or intolerance to prescribed regimens.

Other health consequences of CMV infection are becoming evident; the virus appears to be a critical modifier of both normal and cancerous cell processing. Detection of CMV in blood of presumably immunocompetent hosts (viremia) was linked with outcomes of sepsis and pulmonary complications in CMV-seropositive patients treated in intensive care-units. Virus has also been detected in the brain tumor, glioblastoma multiforme. Although the direct role of CMV in these syndromes is unclear, virus replication may contribute to their natural history, and the potential benefit of anti-viral therapy in these conditions is currently being investigated in clinical trials. At this time there is no approved CMV vaccine despite considerable research efforts to develop one.

### SUMMARY OF THE INVENTION

The present invention is based, at least in part, on the discovery that MDP and/or tri-DAP, or derivatives thereof, when used as pre-treatment can inhibit CMV replication via induction of INF-β pathway, and therefore can be used as vaccine adjuvants to enhance immune response for CMV. As described herein, treatment of human fibroblasts with MDP (NOD2 activator) or tri-DAP (NOD1 activator) before CMV infection, resulted in induction of anti-viral cytokines that inhibited CMV replication. The effect of MDP and tri-DAP was enhanced with prolonged exposure. These bacterial products induced an anti-viral response which was dependent on IFN-β.

Accordingly, in one aspect, the present invention provides methods and compositions useful for preventing human CMV. In one embodiment, the present invention provides an immunogenic composition comprising (a) a live, attenuated or replication-defective cytomegalovirus composition and (b) a NOD1 activator and/or a NOD2 activator. In another embodiment, the present invention provides an immunogenic composition comprising (a) a CMV immunogen composition and (b) a NOD1 activator and/or a NOD2 activator. In certain embodiments, the NOD2 activator is muramyl dipeptide (MDP) or a derivative thereof. In particular embodiments, the NOD1 activator is L-Ala-γ-D-Glu-meso-diamino-pimelic acid (tri-DAP) or a derivative thereof. In another embodiments, the NOD1 activator is γ-D-glutamyl-meso-diaminopimelic acid (γ-D-Glu-mDAP (iE-DAP)). The present invention also provides a pharmaceutical composition comprising a CMV adjuvant, wherein the CMV adjuvant comprises MDP and/or tri-DAP or derivatives thereof. The present invention also provides methods for preventing CMV in a patient comprising the step of administering a composition described herein. Derivatives can include but are not limited to, iE-DAP, 6-D-steraoyl-MDP, GMDP, FK156, M-Tri-DAP, Tri-DAP, and M-Tri-Lys (Girardin et al., 278 J. Biol. Chem. 652-58 (2003)), and TCT (Magalhaes et al., 6 EMBO Rep. 1201-07 (2005)).

In particular embodiments, the live, attenuated, inactivated, killed or replication-defective CMV composition is a CMV vaccine. In other embodiments, the CMV immunogen composition is a CMV vaccine, specifically, a subunit vaccine based on expression of specific CMV immunogens in recombinant or vectored systems. Examples of CMV vaccines include, but are not limited to, Recombinant gB subunit CMV vaccine (Sanofi), DNA cytomegalovirus vaccine ASP0113 (Astellas Pharma), CMV-MVA Triplex vaccine (City of Hope National Medical Center), gB eVLP (VBI Vaccines, Inc.) and CMVpp65-A*0201 (City of Hope National Medical Center). In certain embodiments, the CMV vaccine is a live, attenuated or replication-defective virus vaccine. Examples include Towne vaccine (Med. Coll. VA), recombinants with wild virus (e.g., Towne/Toledo chimera vaccines (MedImmune)), and V160-001 replication-defective vaccine. In other embodiments, the CMV vaccine is a subunit vaccine, based on expression of specific CMV immunogens in recombinant or vectored vaccine systems. Examples include glycoprotein B, PADRE-pp65-CMV fusion peptide, Glycoprotein B/Canarypox vector, pp65 (UL83)/Canartypox vector, gB/pp65/IE1 trivalent DNA vaccine; gB/pp65 bivalent DNA vaccine; and gB/pp65/IE1 alphavirus replicon trivalent vaccine. Non-living CMV vaccines also include DNA plasmids, self-replicating RNA, peptides, dense bodies, virus-like particles and soluble pentamers. Further examples of CMV vaccines can be found in U.S. Pat. No. 8,278,093 (Vical Incorporated); U.S. Pat. No. 7,888,112 (Vical Incorporated); No. 6,726, 910 (City of Hope); and No. 6,544,521 (City of Hope), as well as U.S. Patent Applications Publication No. 20140348863 (Novartis AG); No. 20140220062 (Merck); No. 20150322115 (Redvax GMBH); No. 20150359879 (Redvax GMBH); No. 20150086578 (Virginia Commonwealth University); No. 20140370026 (Princeton University); and No. 20100285059 (Princeton University).

### BRIEF DESCRIPTION OF THE FIGURES

FIG. 1A-1F: MDP treatment inhibits HCMV replication at low MOI. FIG. 1A. HFFs were infected with HCMV-Towne (100 PFU/well) and treated with MDP (10 μg/mL). The number of plaques, counted after 8 days, are average data from two independent experiments (***p<0.001, one-way ANOVA test). FIG. 1B, 1C. HFFs were infected with HCMV-Towne (MOI 0.1 and 1) and treated with MDP. The expression level of indicated proteins was determined at 4 days post infection. FIG. 1D. Dose response curve of MDP. HFFs were infected (MOI 0.1) and treated with MDP at the indicated concentrations of MDP or GCV. The expression of IE1/2, pp65 and β-actin was determined after 4 days. FIG. 1E. HFFs were treated with MDP for 96 h and cell viability was determined using an MTT assay. FIG. 1F. Add-on assay of MDP (10 μg/mL). HFFs were infected (MOI 0.1) and MDP added at the indicated time points. The expression of HCMV proteins was measured at 96 hpi.

FIG. 2A-2G: Effect of MDP on expression of NOD2, IFN-β, and IL8 in HCMV-infected HFFs. FIG. 2A, 2B, 2C. Non-infected or HCMV-infected HFFs (MOI 0.1, and 1 PFU/cell) were non-treated or treated with MDP (10 μg/mL) after infection. Levels of NOD2, IFN-β, and IL8 mRNA were measured by qRT-PCR at 36 hpi. Data shown are mean±SD from triplicate wells of a representative experiment of two independent experiments (*p<0.05, **p<0.01, ***p<0.001, one-way ANOVA test). FIG. 2D, 2E. The expression of proteins downstream of NOD2 in cells infected with MOI 0.1 (D) or 1 (E) was determined by Western blot at 36 hpi. FIG. 2F, 2G. Expression of proteins downstream of NOD2 was measured in the cytoplasmic and nuclear fractions at 36 hpi. Western blot data are from a representative experiment of two independent experiments.

FIG. 3A-3F: Induction of NOD2, IFN-β and IL8 mRNA in HCMV-infected MDP-treated HFFs is NOD2-dependent. FIG. 3A, 3B, 3C. Control (GIPZ) or shNOD2 cells were mock- or HCMV-infected (MOI 0.5) and were either non-treated or treated with MDP (10 μg/mL) for 48 h. NOD2, IFN-β and IL8 mRNA was measured by qRT-PCR. The depicted mRNA expression experiments represent mean±SD from triplicate wells of a representative experiment. FIG. 3D, 3E, 3F. Control (GIPZ) or shNOD2 cells were mock- or HCMV-infected (MOI 3), and either non-treated or treated with MDP (10 μg/mL) for 36 h. NOD2, IFN-β and IL8 mRNA was measured by qRT-PCR. Data shown are mean±SD from triplicate wells of a representative experiment (*p<0.05, **p<0.01, one-way ANOVA test). FIG. 3G. HCMV entry (MOI 3) into control (GIPZ) or shNOD2 cells was determined by Western blot for pp65 at 2 hpi.

FIG. 4A-4K: MDP pretreatment inhibits HCMV replication. FIG. 4A, 4B. HFFs were pretreated with MDP (10 μg/mL) for 72 h followed by infection with HCMV Towne or HCMV TB40 (100 PFU/well). GCV treatment was used after infection. The number of plaques, counted after 8 or 10 days, represent average data from two independent experiments. FIG. 4C, 4D. HFFs were pretreated with MDP (10 μg/mL) for 18 or 72 h followed by infection with pp28 luciferase-recombinant HCMV (MOI 1). Luciferase activity was measured in cell lysates at 96 hpi (1st cycle). Cell free supernatants were collected at 96 hpi for a 2nd cycle infection in fresh HFFs and luciferase activity was measured after 72 hpi. Data are mean±SD from two independent experiments (**p<0.01, ***p<0.001, one-way ANOVA test). FIG. 4E. Supernatants from pp28-luciferase HCMV-infected cells were used to infect fresh HFFs and plaques were counted after 8 days. FIG. 4F, G. HFFs were pretreated with MDP (10 μg/mL) for 18 or 72 h and then infected with HCMV (MOI 1) for 96 h. Cell free supernatants were collected at 96 hpi, and used to infect fresh HFFs (2nd cycle infection). Cell lysates were collected at 72 hpi. The expression of IE1/2, pp65 and (3-actin in lysates from the 1st and 2nd cycle was measured by Western blot. FIG. 4H. HFFs were non-treated or pretreated with MDP (10 μg/mL) for 18 or 72 h and then infected with HCMV (MOI 1) for 96 h. NOD2 expression was determined by Western blot. Representative blots are shown. FIG. 4I. Dose-response of MDP pretreatment at MOI 0.1. HFFs were pretreated for with MDP for 72 h at the indicated concentrations, followed by infection. GCV was added after infection. Luciferase activity was measured at 96 hpi. FIG. 4J. HFFs were non-treated or pretreated with MDP (10 μg/mL) for 72 h, then infected with HCMV (MOI 0.1) for 96 h. Expression of indicated proteins was determined by Western blot. Representative blots are shown. FIG. 4K. HCMV entry into MDP pre-treated (18 h and 72 h) or non-pretreated HFFs was determined by Western blot for pp65 at 2 hpi.

FIG. 5A-5G: MDP-pretreatment of HFFs elicits strong IFN-β response after HCMV infection. FIG. 5A, 5B. HFFs were pretreated with MDP (10 μg/mL) for 72 h, and infected with HCMV (MOI 1) for 24 h. NOD2 and IFN-β mRNA was quantified by qRT-PCR. FIG. 5C. Secreted IFN-β was measured by ELISA. FIG. 5D. HFFs pretreated with MDP (10 μg/mL) for 72 h, were infected with HCMV (MOI of 1) for 24 h. IL8 mRNA was quantified by qRT-PCR. Data represent mean±SD from triplicate wells of a representative experiment of two independent experiments (**p<0.01, ***p<0.001, one-way ANOVA test). FIG. 5E, 5F, 5G. The expression level of NOD2-downstream signaling proteins, those regulating IFN-β expression, and viral proteins were determined in total cell lysates (FIG. 5E), cytoplasmic (FIG. 5F) and nuclear fractions (FIG. 5G). β-actin was used as loading control, Histone H3 was used as control for nuclear proteins. Western blot data are from a representative experiment of three independent experiments.

FIG. 6A-6H: Inhibition of HCMV replication by MDP pretreatment is mediated through IFN-β. FIG. 6A. HFFs were stably transduced with lentivirus expressing either control (GIPZ) or shRNAs against IFN-β (shIFN-β). Cells were infected with the pp28-luciferase Towne strain (MOI 1) for 72 h and levels of IFN-β mRNA were measured by qRT-PCR. Data are average of duplicate values from two independent experiments. FIG. 6B. GIPZ or shIFN-β expressing HFFs were non-treated or treated with MDP and cell viability was determined after 72 h using an MTT assay. Data are average of triplicate values of an experiment. FIG. 6C. Virus entry into lentivirus transduced cells was determined by a Western blot for pp65 at 2 hpi. FIG. 6D. Control or shIFN-β HFFs were non-treated or pretreated with MDP for 72 h followed by infection with HCMV Towne (100 PFU/well). The number of plaques, counted after 8 days, are average data from two independent experiments. FIG. 6E. Control (GIPZ) or shIFN-β HFFs were non-treated or pretreated with MDP for 72 h followed by infection with pp28-luciferase Towne HCMV (MOI 1) for 72 h. Luciferase activity was determined at 72 hpi. Data shown are average of triplicate values from three independent experiments. FIG. 6F. Control or IFN-β-KD HFFs were non-pretreated or pretreated with MDP for 72 h and then infected with HCMV (MOI 0.1) and expression of viral proteins IE1/2, UL44 and pp65 was determined in cell lysates. β-actin was used as a loading control. Representative blots from two independent experiments are shown. FIG. 6G. Control or shIFN-β HFFs were non-pretreated or pretreated with MDP for 72 h and then infected with a clinical isolate of HCMV (MOI 1) and expression of viral proteins IE1/2 was determined at 24 hpi using immunofluorescence microscopy. Representative pictures from two independent experiments are shown. FIG. 6H. HFFs were pretreated with MDP for 72 h followed by infection with pp28-luciferase HCMV. IFN-β receptor blocking antibody (5 μg/mL) was added to infected cells. Expression of viral proteins was measured at 4 days post infection. Representative blots from two independent experiments are shown.

FIG. 7. Tri-DAP treatment after CMV infection results in CMV inhibition. Left: HFFs were infected with pp28-luciferase Towne CMV and treated with Tri-DAP (10 μg/mL). Luciferase activity and CMV protein expression was measured by luminescence and Western blot at 72 hpi. Right: Supernatants from first cycle were used for infection of fresh HFFs and a plaque assay was performed at day 10 post infection.

FIG. 8A-8D: Tri-DAP pretreatment inhibits CMV replication. FIG. 8A. HFFs were pretreated with Tri-DAP (10 μg/mL) for 18 or 72 h followed by infection with pp28-luciferase recombinant CMV (MOI 1). Luciferase activity was measured in cell lysates at 96 hpi (1st cycle). Cell free supernatants were collected at 96 hpi for a 2nd cycle infection in fresh HFFs and luciferase activity was measured after 72 hpi. Data are mean±SD from two independent experiments. FIG. 8B. Western blot analysis of CMV proteins from FIG. 8A. FIG. 8C. HFFs were pretreated with Tri-DAP for 72 h followed by infection with CMV Towne (100 PFU/well). The number of plaques, counted after 10 days, represent average data from two independent experiments. FIG. 8D. HFFs were pretreated with Tri-DAP for 72 h and cell viability was determined using an MTT assay. Results from a representative experiment of two independent experiments are shown.

FIG. 9A-9D: NOD1 activator, iE-DAP, inhibits MCMV replication. BALB/c mice (3-4 weeks) were pretreated with iE-DAP. Blood was collected 4 h after the second dose of iE-DAP and RANTES levels were measured by ELISA in serum samples (A). At 14 days' post infection blood was collected for gB real-time PCR (B) and plaque assays were performed from salivary glands, liver and spleen (C). GCV was used after infection at 10 mg/kg twice daily for 5 days (D). Data are presented as Mean±SE of PFU/100 mg of tissue homogenate.

FIG. 10: Survival curve of CMV-infected NOD2 knockout mice compared to wildtype (WT) mice. 4-week old BALB/c mice were infected with MCMV at 10⁶ PFU/mice. In mice that lack NOD2, CMV infection is associated with worse survival.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### Limited Treatment Options for CMV.

Treatment for CMV disease is based on a limited number of drugs, all of which inhibit the viral DNA polymerase. Despite suppressing CMV replication, the systemic anti-CMV agents cause intolerable toxicities to the bone marrow (ganciclovir-GCV) and kidneys (foscarnet and cidofovir) and select for resistant viral mutants during prolonged courses of therapy, for which there are no treatment alternatives. Until recently, intravenous GCV was the only drug approved for congenital CMV infection with central nervous system involvement, based on a phase III clinical trial in which prevention of hearing loss was documented in treated children. This clinical trial demonstrated that anti-viral therapy has an important role in the outcome of congenital CMV-associated hearing loss. Recent data from a phase III clinical trial of oral valganciclovir (the valyl-ester prodrug of GCV) in congenitally-infected children reveal that 6 months therapy may have a better neurological outcome than 6 weeks therapy, but GCV-resistant mutants emerge. GCV-resistant strains are also detected in adults treated with prolonged courses of GCV. Widespread use of a limited number of drugs eventually leads to development of drug resistant strains. Thus, new strategies for CMV therapy that involve novel targets and/or novel therapeutic concepts are urgently needed. Prevention of CMV infection or disease using a vaccine would be ideal, but despite significant research effort, a CMV vaccine is not yet available. The best vaccine strategy and the appropriate adjuvants are still under intense investigation.

Intrinsic Immunity to CMV.

Despite recognized episodes of CMV reactivation in immunosuppressed CMV seropositive patients, CMV disease occurs only in a subset of these individuals, suggesting a role for host genetics in disease development. In addition, while CMV infection is common, 10-15% of elderly individuals remain CMV negative. Seronegativity may reflect lack of exposure to the virus; an alternative explanation is that host genetics contributes to the susceptibility to CMV. Indeed, genetics can influence susceptibility to infection and cytokine production by the innate immune system. Individuals can be either high or low inflammatory responders. Among human genes, four main classes of pattern recognition receptors (PRRs) have been described: 1) The best-known are the Toll-like receptors (TLRs), which are localized at the cell surface, or within endosomes, and recognize microbial structures from Gram-positive and Gram-negative bacteria, RNA and DNA viruses, fungi, and protozoa; 2) Nucleotide-binding oligomerization domain and leucine rich repeat containing receptors (NODs or NLRs); 3) Retinoic-acid-inducible gene I (RIG-I)-like receptors (RLRs); and 4) DNA sensors: absent in melanoma 2 (AIM2) and cyclic GMP-AMP synthetase (cGAS). NLRs, RLRs, AIM2 and cGAS sense pathogens in intracellular compartments. To date, NLRs have been mostly reported to be activated in response to bacterial products, whereas RLRs typically sense viruses. We reported for the first time that NOD2 is induced by CMV, activates downstream signaling pathway which in turn result is suppression of CMV replication. Our preliminary data also support a role for NOD1 in CMV replication. We propose that modulation of antiviral responses downstream of NOD1 and NOD2 may provide a new strategy for CMV prevention and therapeutics.

NODs Recognize Pathogen Associated Molecular Patterns and Initiate Specific Innate Immune Responses.

NOD1 and NOD2 are the most studied members of the NLR family. They are structurally similar. NOD1 contains an amino-terminal region consisting of protein-protein interaction domain known as the caspase-recruitment domain (CARD), an intermediary NOD that is required for nucleotide binding and self-oligomerization; and a carboxy-terminal leucine-rich repeat (LRR) domain that detects conserved microbial patterns and modulates NLR activity. NOD2 contains two CARD domains. Mutations in NOD2 are strongly associated with Crohn's disease, whereas mutations in NOD1 have been linked with asthma and atopic eczema. NOD1 and NOD2 are highly expressed in monocytes, macrophages, and dendritic cells. NOD1 is also expressed in epithelial cells, and NOD2 expression is induced by inflammatory signals in these cells. Unlike NOD2, polymorphisms in NOD1 have not been robustly linked with increased susceptibility to Inflammatory Bowel Diseases. However, NOD1 is highly expressed in intestinal epithelial cells and probably plays an important role in regulating host responses to the normal gut microbiota and to enteric pathogens in these cells. Intestinal expression of NOD2 may also be regulated by the microbiota; germfree mice had lower NOD2 expression that was restored upon colonization with commensal bacteria. Work from our laboratory reveals that NOD2 is significantly induced in CMV-infected human foreskin fibroblasts (HHFs), while NOD1 induction is modest. Both NOD1 and NOD2 play a role in restricting CMV replication. NOD1 recognizes a fragment of peptidoglycan (PGN) containing the dipeptide γ-d-glutamyl-meso-diaminopimelic acid (iE-DAP or Tri-DAP) produced by Gram-negative and some Gram-positive bacteria. NOD2 recognizes muramyl dipeptide (MDP) present on most types of PGN. More recently N-glycolyl muramyl dipeptide from mycobacteria was reported to activate NOD2.

The Role of NOD1 and NOD2 in Suppression of CMV Replication.

NOD1 and NOD2 are well-established as bacterial sensors. Their ability to recognize viruses is starting to become evident. The single stranded (ssRNA) respiratory syncytial virus (RSV) was reported to activate NOD2. We reported that NOD2 is induced by CMV, activates antiviral cytokines responses resulting in suppression of virus replication.

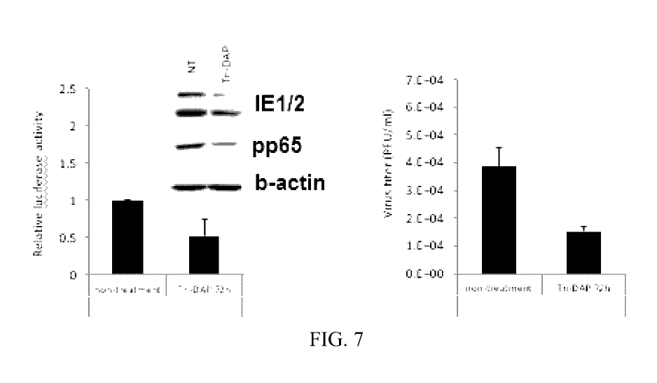

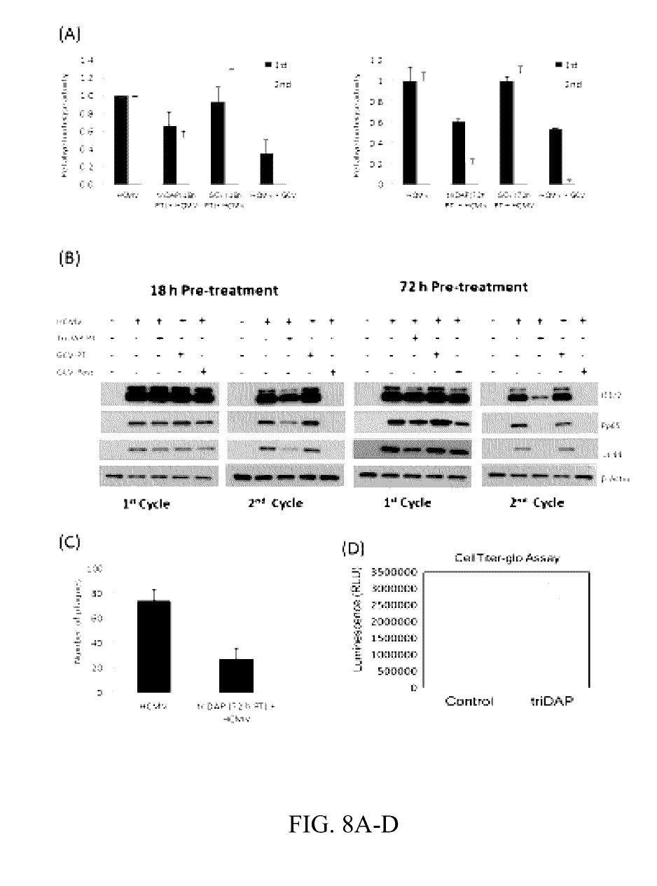

MDP (which binds to and activates NOD2) inhibits CMV replication in a dose and time-dependent manner (FIG. 7). In addition, induction of NOD1 by Tri-DAP results in virus inhibition (FIG. 8). Since both NOD1 and NOD2 require the kinase RIPK2 for their downstream signaling activities, investigations are ongoing in our laboratory to delineate the role and balance of NOD1 and NOD2 in CMV inhibition. Clearly, MDP and Tri-DAP generate an antiviral environment and thus offer novel candidates for CMV therapeutics and prevention.

MDP and Tri-DAP Act as Adjuvants for Prevention of Infectious Diseases.

MDP was discovered to be the minimal structure required for the efficacy of Freund's Complete Adjuvant (FCA), one of the most potent and widely used adjuvants in animal models. Composed of heat-killed mycobacterial components in an oil emulsion, FCA elicits strong humoral and cellular immune responses. Unfortunately, its toxicity hampers its use in a clinical setting. A search for smaller, biologically active components in FCA resulted in the discovery of a tripeptidemonosaccharide. A series of similar peptide-monosaccharides were synthesized and tested in rabbits for adjuvant activity through their ability to elicit immunoglobulin production. These peptides included MDP and Tri-DAP-containing peptides. MDP was the smallest compound found to elicit adjuvant activity and could thus replace FCA for its ability to induce humoral and cellular activity. However, it did not induce immunoglobulin production as it is a pure adjuvant lacking the antigens contained in the FCA complex. MDP and its derivatives have a tremendous therapeutic potential. For example, murabutide (MB), is a synthetic immunomodulator derived from MDP that enhances non-specific resistance to bacterial and viral infections without fever and decreases the lethality of LPS in mice. It has also been observed to synergize with antiviral and anti-inflammatory cytokines such as IFN-α as well as increase the anti-tumor effects of IFN-α and IL-2 in mouse models. Most importantly, MB regulates cytokine production without dramatically inducing proinflammatory mediators. MB significantly inhibits HIV-1 replication in acutely infected monocyte-derived macrophages and dendritic cells. Efforts have already been made to develop other similarly MDP-derived drugs. Macrophages activated by a liposome-encapsulated immunomodulator (MTP-PE, a MDP derivative) or MDP conjugated by PolyG (a 10-mer polyguanylic acid), have resulted in tumoricidal activity. These derivatives and others can be identified through high throughput screening (HTS), as proposed in our application.

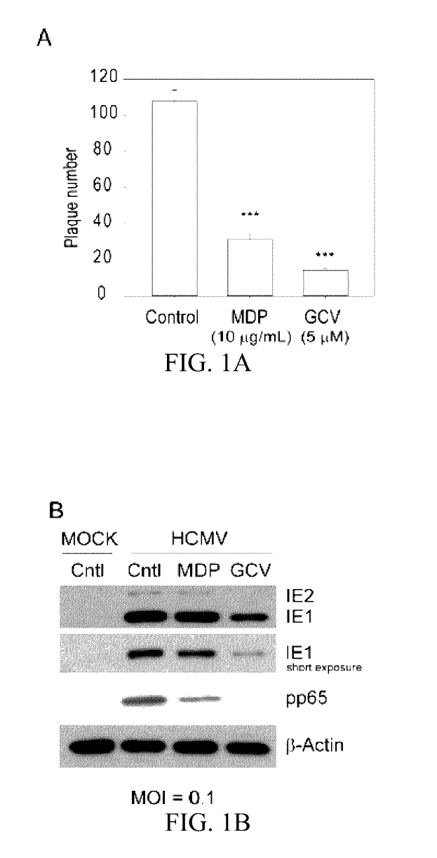

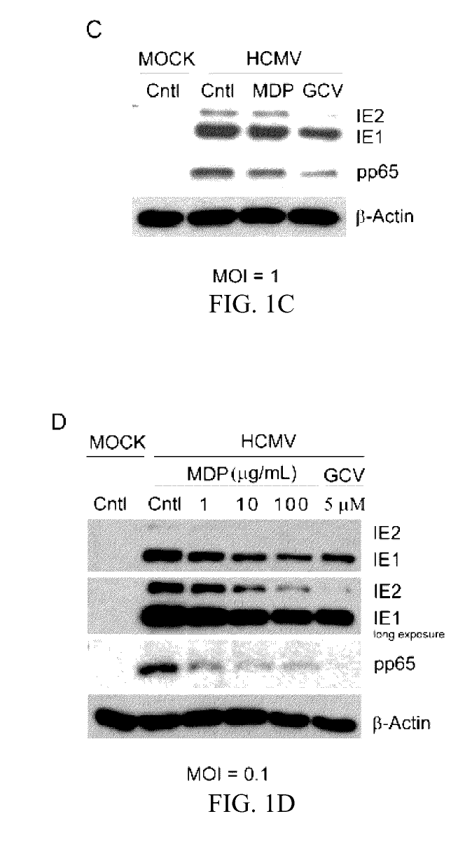

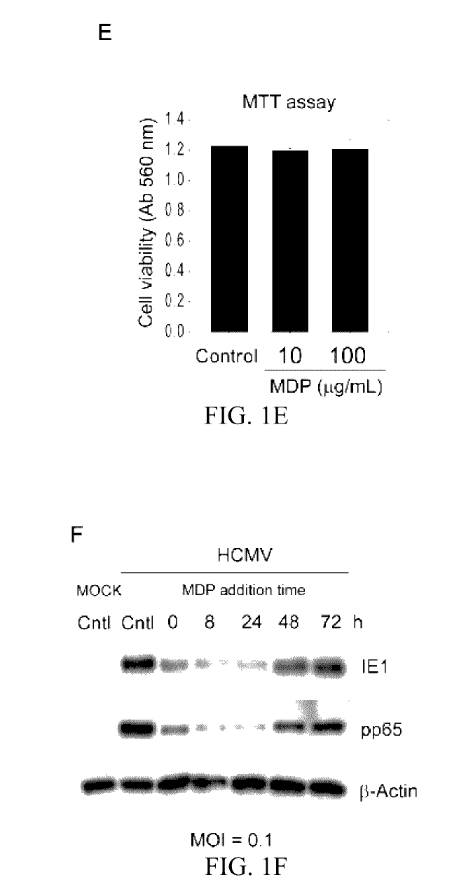

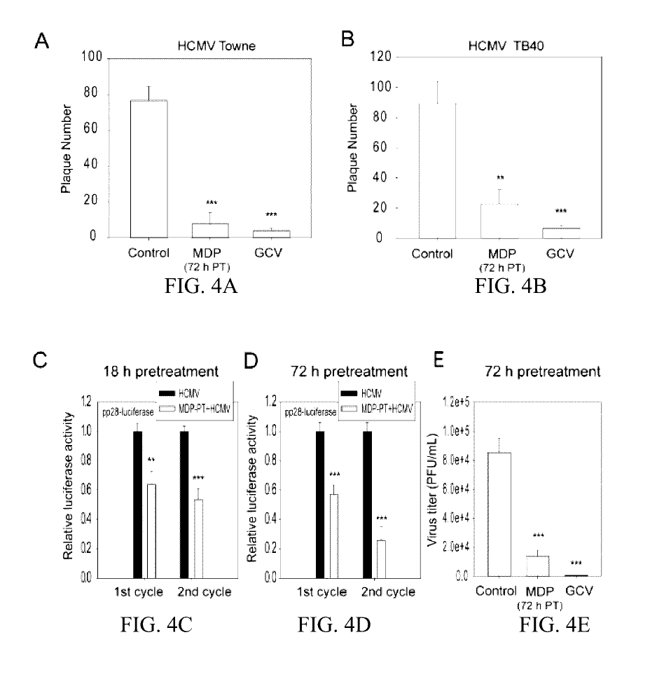

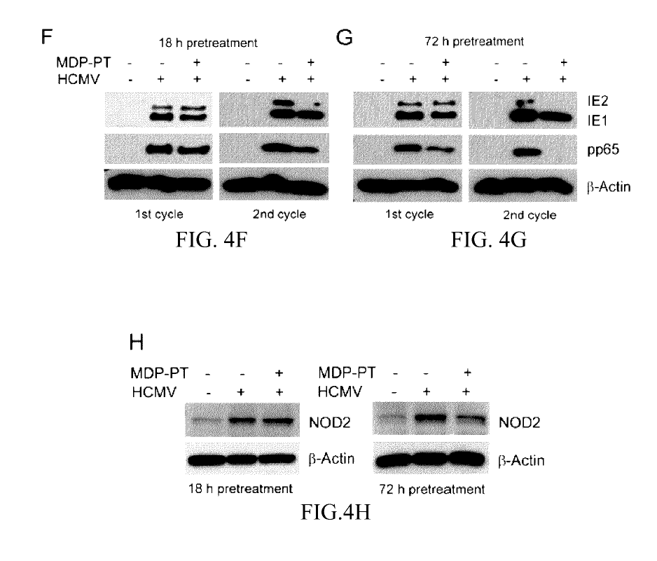

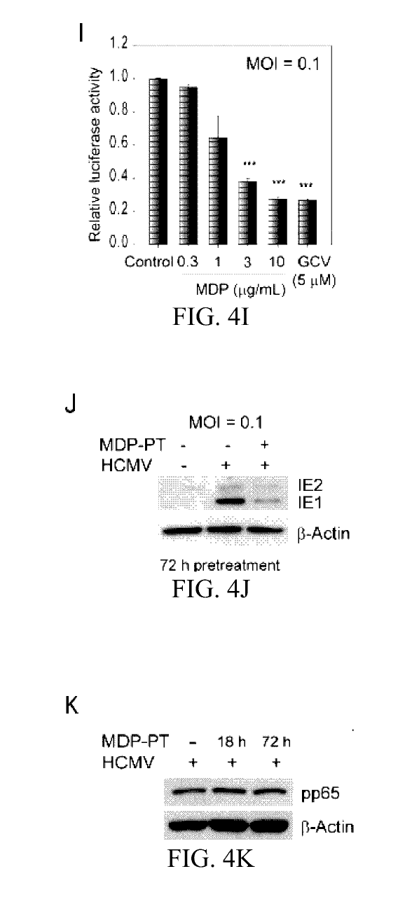

As described herein, the present inventor proposes that MDP and or Tri-DAP can serve as novel adjuvants for CMV vaccines as prevention strategy, since we found that treatment of HFFs before infection could elicit a strong antiviral response and result in virus inhibition (FIGS. 4 and 8). In addition, treatment with MDP or Tri-DAP following CMV infection can also suppress virus replication suggesting a therapeutic role for MDP, tri-DAP or both (by modulating innate immune responses) as adjunct therapy to currently used antivirals (FIGS. 1 and 7). A combination therapy consisting of agents that directly target virus proteins together with agents that modulate innate immune responses to CMV would offer a novel strategy for CMV therapeutics.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Modulation of Innate Immune Response as a Strategy for Vaccine Development and Therapeutics for Cytomegalovirus

### Materials and Methods

Reagents and Chemicals.

Muramyl dipeptide (MDP), a peptidoglycan moiety present in Gram positive and Gram negative bacteria which binds to and activates NOD2, was obtained from Invivogen (San Diego, Calif.) and dissolved in endotoxin free water. A stock of 10 mg/mL was prepared and stored at −20° C. Ganciclovir (Sigma Aldrich, St. Louis, Mo.) was dissolved in distilled water and stored at −80° C.

Cell Culture and Viruses.

Human Foreskin Fibroblasts (HFFs) passage 12-16 (ATCC, CRL-2088™) were grown in Dulbecco's Modified Eagle Medium (DMEM) containing 10% fetal bovine serum (FBS) (Gibco, Carlsbad, Calif.) in a 5% CO₂ incubator at 37° C. The generation of NOD2-KD HFFs (HFF-shNOD2) and control cells (HFF-GIPZ) was reported. One day prior to infection or treatment, 8×10⁴ cells were seeded into each well of 12-well tissue culture plates. Multiplicities of infection were specified for each experiment. The Towne HCMV strain was obtained from ATCC (VR-977). The pp28-luciferase Towne strain which expresses luciferase under the control of the pp28 late promoter has been described. Luciferase activity was measured at 72 or 96 hours post infection (hpi) using the Glomax-Multi+ Detection System (Promega, Madison, Wis.). In second cycle replication assays cell free supernatants from HFFs infected with pp28-luciferase Towne were collected 4 days post completion of first replication cycle (5-10% of total volume/well), and used for infection of fresh HFFs seeded into 12-well plates. Cell lysates were collected after 72 h for luciferase activity. Supernatants from the first cycle were also used to measure virus titer in 12-well plates and plaques were counted at day 8 post infection. A purified preparation of the pp28-luciferase Towne strain was prepared by ultracentrifugation using sucrose gradient. The HCMV TB40 strain was obtained from ATCC (VR-1578). Clinical isolates of HCMV were obtained from the microbiology laboratory at Johns Hopkins Hospital with no identifiers that could be linked to a patient. The Johns Hopkins School of Medicine Office of Human Subject Research Institutional Review Board (IRB-X) determined that the research qualified for an exemption.

Plaque Assay.

A plaque reduction assay was performed in HFFs to determine the effects of MDP treatment either before or after infection on virus replication. HFFs were seeded into 12-well plates (2×10⁵ cells/well) and infected with Towne HCMV at approximately 100 plaques/well. After 90 minutes, media were aspirated, and DMEM containing 0.5% carboxymethyl-cellulose (CMC), 4% fetal bovine serum (FBS), and MDP at final concentration of 10 μg/mL were added into duplicate wells. In pretreatment experiments HFFs were first treated with MDP (10 μg/mL) for 72 h after which infection with the Towne or TB40 strains was carried out at approximately 100 plaques/well. After 90 minute adsorption, media were aspirated, and DMEM containing 0.5% CMC and 4% FBS were added into duplicate wells. After incubation at 37° C. for 8 days (Towne) or 10 days (TB40) the overlay was removed and plaques were counted after crystal violet staining.

Add-on Assay.

HFFs were infected with HCMV Towne (MOI 0.1). At 0, 8, 24, 48 and 72 hpi, the medium was replaced with fresh medium containing MDP (10 μg/mL). A Western blot for IE1/2, pp65, and β-actin was performed at 96 hpi.

Cellular Toxicity.

MTT assay (Sigma-Aldrich) was performed to rule out any cytotoxicity induced by MDP treatment. Cells (HFFs and transduced GIPZ/shIFNβ-HFFs as described below) were pretreated with MDP for 96- and 72 h, respectively and 20 μl/well of MTT [3-(4, 5-Dimethyl-2-thiazolyl)-2, 5-diphenyl-2H-tetrazolim bromide, 5 mg/mL in phosphate buffered saline (PBS)] was added to each well. After shaking plates at 150 rpm for 5 minutes the plates were incubated at 37° C. for 3 hours. Conversion of yellow solution to dark blue formazan by mitochondrial dehydrogenases of living cells was quantified by measuring absorbance at 560 nm.

Lentivirus-mediated KD of IFN-β.

Human GIPZ lentiviral shRNAmir constructs (Open Biosystems, Huntsville, Ala.) were used for IFN-β KD in HFFs. Five clones (V2LHS_238693, V3LHS_355340, V3LHS_355342, V3LHS_355343, V3LHS_355344) targeting different regions of IFN-β mRNA were tested and the clone (V3LHS_355343) with the best KD efficiency was selected for further experiments. GIPZ non-targeting control plasmid was used to rule out non-specific effects of shRNAmir constructs. Individual shRNAmir constructs were packaged using lentivirus as described previously. Briefly, 21 μg of gag/pol, 7 μg of vesicular stomatitis virus glycoprotein, and 7 μg of shRNAmir plasmids were transfected into HEK293 cells using calcium phosphate method. After 48 h the packaged lentivirus particles were concentrated from the medium. The supernatant was filtered and centrifuged at 1750 g for 30 min at 4° C. in Amicon Ultra (Ultracel 100 k, Millipore). After centrifugation, 2 ml of cold PBS was added and the tubes were centrifuged again for 20 min at 4° C. The concentrated virus was stored at −80° C. until used. Lentivirus particles containing shRNAmir were transduced into HFFs. 0.5×10⁶ cells were plated onto T-25 flask and 40 μl of concentrated virus and Polybrene (final concentration, 8 μg/mL) were added to the cells, and incubated for 4 h. Following transduction puromycin (2 μg/mL) was added to select for stably transduced cells.

RNA Isolation and Real-Time Quantitative Reverse Transcriptase (qRT)-PCR.

Total RNA was isolated from cells using RNAeasy Mini kit (Qiagen, Georgetown, Md.) according to manufacturer's instructions. RevertAid first strand cDNA synthesis kit (Fermentas life sciences, Cromwell Park, Md.) was used to synthesize first strand cDNA from total RNA using oligo-dT primers. Negative reverse-transcriptase (−RT) reactions were included to ensure the specificity of qRT-PCR reactions. Synthesis of first strand cDNA from mRNA template was carried out at 42° C. for 1 h. Quantitative RT-PCR was performed using specific primers and SYBR green (Fermentas life science) with two-step cycling protocol (95° C. for 15 s, 60° C. for 1 min). All reactions were performed in triplicates and GAPDH was used as internal control. The expression level of the tested genes was normalized to the expression of GAPDH. The primer sequences were:

(SEQ ID NO: 1)

| | |
|---|---|
| NOD2-Forward, | 5′-GCCACGGTGAAAGCGAAT-3′, |

(SEQ ID NO: 2)

| | |
|---|---|
| NOD2-Reverse, | 5′-GGAAGCGAGACTGAGCAGACA-3′, |

(SEQ ID NO: 3)

| | |
|---|---|
| IFN-β-Forward | 5′-GATTCATCTAGCACTGGCTGG-3′, |

(SEQ ID NO: 4)

| | |
|---|---|
| IFN-β-Reverse, | 5′-CTTCAGGTAATGCAGAATCC-3′, |

(SEQ ID NO: 5)

| | |
|---|---|
| IL8-Forward, | 5′-TGCAGCTCTGTGTGAAGGTGCAGT-3′, |

(SEQ ID NO: 6)

| | |
|---|---|
| IL8-Reverse, | 5′-CAGTGTGGTCCACTCTCAATCACTC-3′, |

(SEQ ID NO: 7)

| | |
|---|---|
| GAPDH-Forward | 5′-TTGGTATCGTGGAAGGACTC-3′ |
| and | |

(SEQ ID NO: 8)

| | |
|---|---|
| GAPDH-Reverse, | 5′-ACAGTCTTCTGGGTGGCAGT-3′. |

SDS-Polyacrylamide Gel Electrophoresis and Immunoblot Analysis.

Cell lysates containing equivalent amount of proteins were mixed with an equal volume of sample buffer (125 mM Tris-HCL, pH 6.8, 4% SDS, 20% glycerol and 5% β-mercaptoethanol) and boiled at 100° C. for 10 min. Denatured proteins were resolved in Tris-glycine polyacrylamide gels (10-12%) and transferred to polyvinylidine difluoride (PVDF) membranes (Bio-Rad Laboratories, Hercules, Calif.) by electroblotting. Membranes were incubated in blocking solution [5% w/v non-fat dry milk and 0.1% Tween-20 in PBS (PBST)] for 1 h, washed with PBST, and incubated with antibody at 4° C. overnight. Membranes were washed with PBST and incubated with horseradish peroxidase-conjugated secondary antibodies in PBST for 1 h at room temperature. Following washing with PBST, protein bands were visualized by chemiluminescence using SuperSignal West Dura and Pico reagents (Pierce Chemical, Rockford, Ill.). Antibodies for HCMV proteins were used at 1:2000 and included: mouse monoclonal anti-HCMV IE1 & IE2 (MAB810, Millipore, Billerica, Mass.), mouse monoclonal anti-HCMV UL83 (pp65, Vector Laboratories Inc., Burlingame, Calif.), and mouse monoclonal anti-pp52 (UL44, Santa Cruz Biotechnology, Santa Cruz, Calif.). Rabbit polyclonal anti human-NOD2 antibody (H-300, 1:2000), rabbit polyclonal anti-RIPK2 (1:5000), mouse monoclonal anti-NF-KB (p65, 1:1000), rabbit polyclonal anti-IRF3 (1:1000) and mouse monoclonal anti-IRF7 (1:1000) were from Santa Cruz. Rabbit monoclonal anti-Histone H3 (1:2000), rabbit monoclonal anti-pERK1/2 (1:5000), mouse monoclonal anti-total ERK (1:2000), rabbit monoclonal anti-pIκBα (1:2000), mouse monoclonal anti-IκBα, rabbit monoclonal anti-pTBK1 (Ser172), and rabbit monoclonal anti-TBK1 (1:1000) were from Cell Signaling Technology, Beverly, Mass. Horseradish peroxidase (HRP)-conjugated anti-rabbit IgG was from Cell Signaling. Horseradish peroxidase (HRP)-conjugated anti-mouse IgG was from GE Healthcare (Waukesha, Wis.). Mouse monoclonal anti-human β-actin 1:5000 was from Sigma. Mouse monoclonal anti-human IFNα/β receptor chain 2 antibody (EMD Millipore Corporation, CA) was used at 5 μg/mL to block the IFN-β receptor. Anti-mouse IgG (Santa Cruz) was used as control in the IFN-β receptor blocking experiment.

ELISA.

The effect of MDP pretreatment on levels of secreted IFN-β in supernatants from non-infected or HCMV-infected HFFs was measured by IFN-β specific ELISA kit (PBL Assay Science, Piscataway, N.J.).

Preparation of Cytoplasmic and Nuclear Extracts.

Cytoplasmic and nuclear fractions were isolated from HCMV-infected or mock-infected HFFs at 24 hpi as previously reported. Briefly, cells were washed twice with ice-cold PBS and resuspended on ice for 15 min in buffer A containing 10 mM HEPES (pH 7.9), 10 mM KCl, 0.1 mM EDTA, 1 mM dithiotheitol (DTT), protease and phosphatase inhibitors. Cells were then lysed by adding 0.1% NP40 and cytosolic supernatants were obtained by centrifugation at 10,000 rpm for 30 sec. Crude nuclei were washed twice with buffer A to prevent cytoplasmic contamination, and the nuclear proteins were extracted by resuspending cell pellets with buffer C containing 20 mM HEPES (pH 7.9), 400 mM NaCl, 1 mM EDTA, 1 mM DTT, protease and phosphatase inhibitors. The mixture was incubated for 15 min with vigorous shaking on rocker at 4° C. and then centrifuged at 14,000 rpm at 4° C. for 10 min to obtain the nuclear proteins. Protein concentration was determined using BCA protein assay reagent kit (Pierce Chemical, Rockford, Ill.).

Immunofluorescence Staining for HCMV IE1/2.

GIPZ control and shIFN-β HFFs were seeded into 96-well plate (1×10⁴ cells/well) and pretreated with MDP (10 μg/mL) for 72 h. Cells were then infected with a clinical isolate of HCMV at MOI 1. Twenty-four hours after infection, cells were fixed with methanol/acetone (1:1) at −20° C. for 10 min. Cells were then blocked with 7.5% BSA for 30 min. and incubated for 1 h with anti-human CMV IE1&2 antibody (MAB810) (1:500 in 0.5% BSA). After 3 washes with PBS, cells were incubated with FITC-anti mouse antibody (F8521, Sigma, 1:500, in 0.5% BSA) for 1 h. Then cells were washed 3 times with PBS and counterstained with propidium iodide (Invitrogen) for nuclear staining. Images were taken using Nikon Eclipse TS100 microscope.

Quantification Densitometry and Statistical Analysis.

Quantitative analysis of proteins detected by immunoblotting was performed using ImageJ 1.48v software (NIH) by determining relative band intensities of individual bands. Statistical analysis was performed using one-way ANOVA comparison between different groups with significance value set at p<0.05.

Results

MDP Treatment Restricts HCMV Replication at Low MOI

The Effect of MDP on virus replication was investigated. HFFs were infected with HCMV Towne at 100 PFU/well and treated with MDP (10 μg/mL) or ganciclovir (GCV, 5 μM). MDP treatment reduced plaque number (FIG. 1A) and expression of HCMV proteins, as determined by Western blot (FIG. 1B). The effect of MDP treatment on the expression of IE1/2 and pp65 was MOI dependent (FIGS. 1B&C). At 96 hpi MDP treatment resulted in a significant decrease (approximately 75%) in pp65 expression (MOI 0.1), but the reduction in pp65 expression was modest (40%) at a higher MOI. Changes in IE1 expression by MDP were also MOI-dependent. MDP suppressed HCMV replication (MOI 0.1) in a dose-dependent manner (FIG. 1D), an effect that was not secondary to cellular toxicity, since an MTT assay performed during the same time point revealed no toxicity at any of the concentrations used (FIG. 1E). Addition of MDP at different time points after infection revealed that IE1 and pp65 expression was significantly reduced when MDP was added after 8 h and before 48 hpi, suggesting its inhibitory effect occurred mainly at an early stage of virus replication (FIG. 1F). We previously reported that induction of NOD2 was relatively low before 24 hpi. The add-on assay (FIG. 1F) correlates with these findings, indicating that the most effective time for MDP activity is between 8 and 48 hpi.

MDP Treatment in HCMV-Infected HFFs Induces NOD2, IFN-β and IL8.

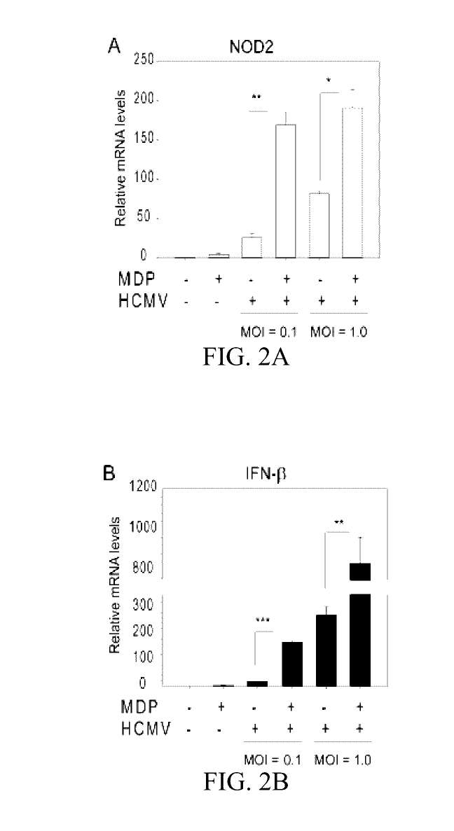

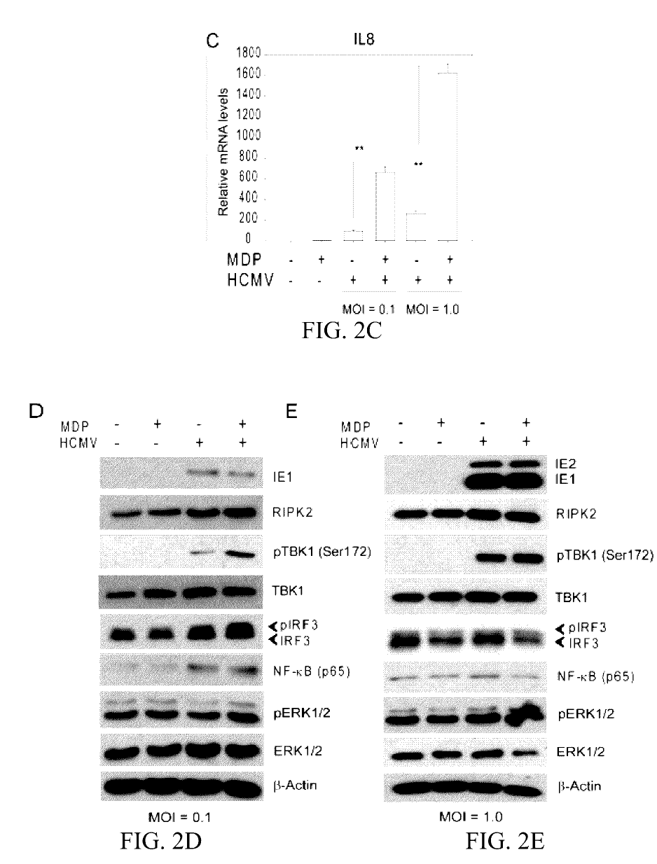

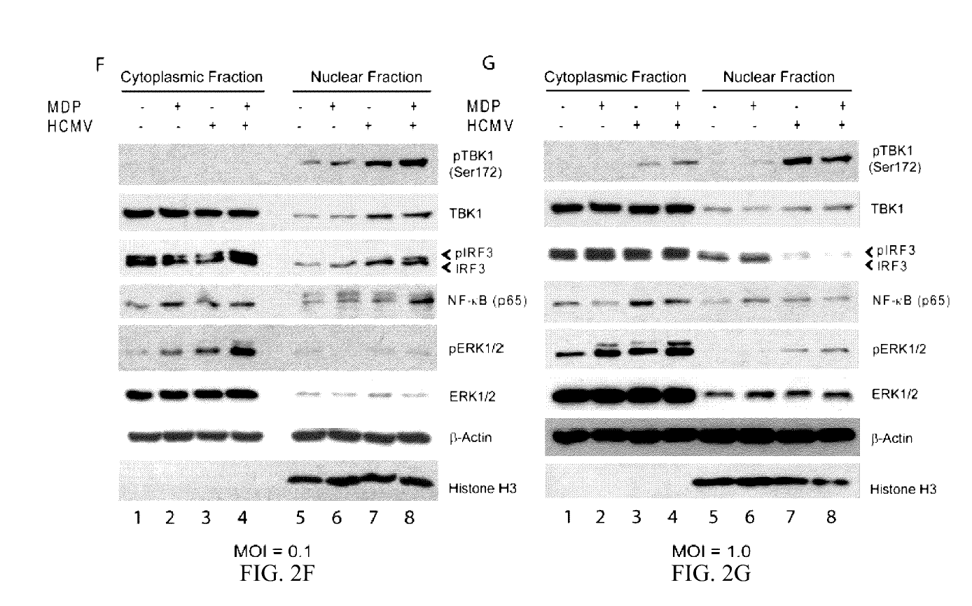

Since MDP inhibited HCMV replication, its effects on the expression of NOD2, IFN-β and IL8 mRNA was investigated in non-infected and HCMV-infected HFFs. We previously reported on significant induction of NOD2 expression starting at 24 hpi and thereafter and on NOD2-dependent induction of IRF3 by HCMV. HFFs were infected with the Towne strain (MOI 0.1, and 1) and after 90 minutes treated with MDP (10 μg/mL). The expression of NOD2, IFN-β and IL8 mRNA was measured at 36 hpi (FIGS. 2A, B&C). This time point was chosen based on our previous report showing significant NOD2 induction at 24 hpi. MDP treatment of HCMV-infected cells resulted in enhanced induction of NOD2 mRNA, compared to NOD2 induction by infection or MDP treatment alone (FIG. 2A). In non-infected HFFs, MDP treatment resulted in 4-fold induction of NOD2 mRNA. NOD2 induction by MDP was more significant at a lower MOI (FIG. 2A, 6.5-fold, MOI 0.1) than at higher MOI (2.5-fold, MOI 1). IFN-β expression was induced by 4-, 15- and 300-fold with MDP, HCMV at MOI 0.1 and 1.0, respectively, compared to its expression in non-infected cells. MDP treatment after low MOI (0.1) resulted in significant induction of IFN-β mRNA compared to infection-only (170- vs 15-fold), an effect that was modest at MOI of 1 (800- vs 300-fold induction, FIG. 2B). These results demonstrate an enhanced effect of MDP in HCMV-infected cells in inducing IFN-β expression at low MOI. IL8 expression was induced 3-, 90- and 260-fold by MDP and HCMV (MOI 0.1 and 1), respectively. MDP treatment after infection further increased (6-7 fold) IL8 mRNA compared to MDP or HCMV alone, irrespective of the MOI used (FIG. 2C).

Enhanced Activation of NOD2 Downstream Signaling Pathways in HCMV-Infected MDP-Treated HFFs.

Upon detection of MDP, NOD2 binds to the RIPK2 via CARD-CARD homophilic interactions, a step required in order for downstream signaling to proceed (30). RIPK2 is a critical kinase downstream of NOD2, since in RIPK2 deficient cells, NOD2 signaling is abolished. We previously reported that overexpression of NOD2 or RIPK2 restricted HCMV replication. We therefore investigated the effect of MDP on expression of NOD2 downstream signaling proteins in HCMV-infected cells at 36 hpi (FIGS. 2D&E). RIPK2 expression was increased by 2.5 fold after infection (MOI 0.1, FIG. 2D, lanes 1&3) and further increased in infected MDP-treated HFFs (lanes 3&4). MDP treatment resulted in increased phosphorylation of TANK-binding kinase (TBK1) at MOI 0.1, but levels of total TBK1 were unchanged (FIG. 2D). The expression of total and phospho IRF3 (pIRF3) were also increased in MDP-treated HCMV-infected cells, an effect observed again only at MOI 0.1 (FIG. 2D, lanes 3&4), but not at MOI 1 (FIG. 2D and FIG. 1E, respectively). The increased phosphorylation of TBK1 and IRF3 was observed in the nuclear fraction (FIG. 2F, MOI 0.1). At higher MOI, MDP treatment did not induce pTBK1 or pIRF3 (FIGS. 2E, G).

NF-κB (p65) expression increased with infection (FIG. 2D, lanes 1&3), and although MDP did not induce NF-KB in non-infected or HCMV-infected cells in total cell lysates, at low MOI, MDP treatment of infected cells resulted in increased nuclear localization of NF-κB (FIGS. 2F&G). Only at MOI 0.1 NF-KB expression was increased with MDP treatment in the nuclear fraction (FIG. 2F, lanes 5&8). The expression of pERK in total lysates was not induced by HCMV at 36 h, and treatment with MDP only modestly increased pERK2 (p42 MAPK) in infected cells (FIGS. 2D and 2E, lanes 3&4). Since HCMV was reported to induce pERK at an earlier time point (also observed by us at 4 hpi, (data not shown)), these results suggest that MDP may prolong pERK2 expression. In the cytoplasmic fraction MDP induced pERK in non-infected cells (FIGS. 2 F and 2G lanes 1&2). At MOIs 0.1 and 1, MDP induced pERK2 expression of infected cells to similar level as in non-infected HFFs. (FIGS. 2F&G). Since MDP did not inhibit HCMV at MOI 1 (FIG. 1C), the MDP-induced changes in pERK (FIGS. 2 F and 2G) may not contribute directly to HCMV inhibition but rather independently regulate expression of cytokines, such as IL8.

The expression of RIPK2 was induced at MOI 0.1 and 1 (FIG. 2D, 2E lanes 1&3), and MDP treatment further induced RIPK2 expression at MOI 0.1 (FIG. 2D lanes 3&4). However, RIPK2 expression as well as that of pTBK1, pIRF3 and NF-KB were unchanged after MDP treatment at MOI 1 (FIG. 2E, for high MOI). These results suggest that at high MOI HCMV could overcome the MDP-induced changes in these signaling proteins, and that the downstream effects of MDP on TBK1 and IRF3 phosphorylation are MOI-dependent. The effects of MDP in HCMV-infected cells were tested at an early time (4 hpi). There was only 2-fold increase in IFN-β and IL8 mRNA with MDP treatment, and no changes were observed in the expression of signaling proteins downstream of NOD2, irrespective of MOI used (data not shown).

Activation of the IFN-β Pathway by MDP at Low MOI is NOD2-Dependent.

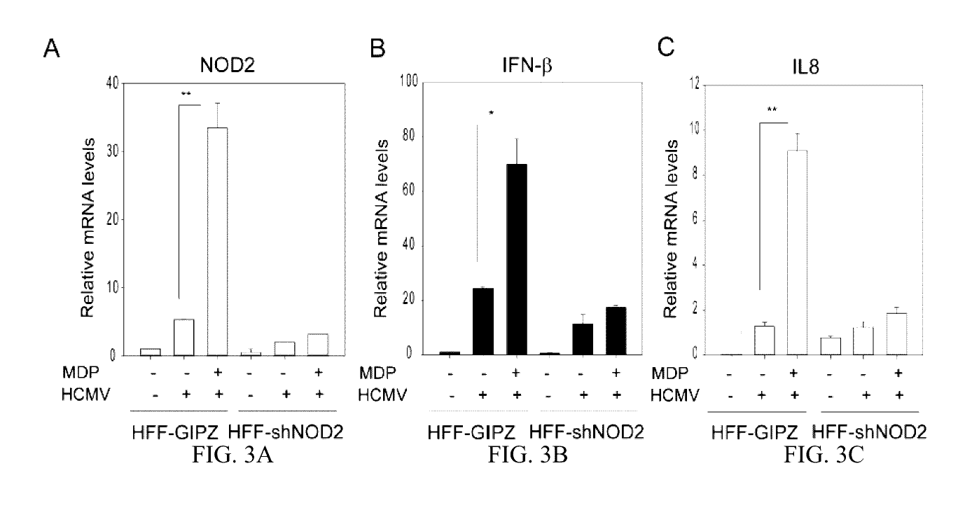

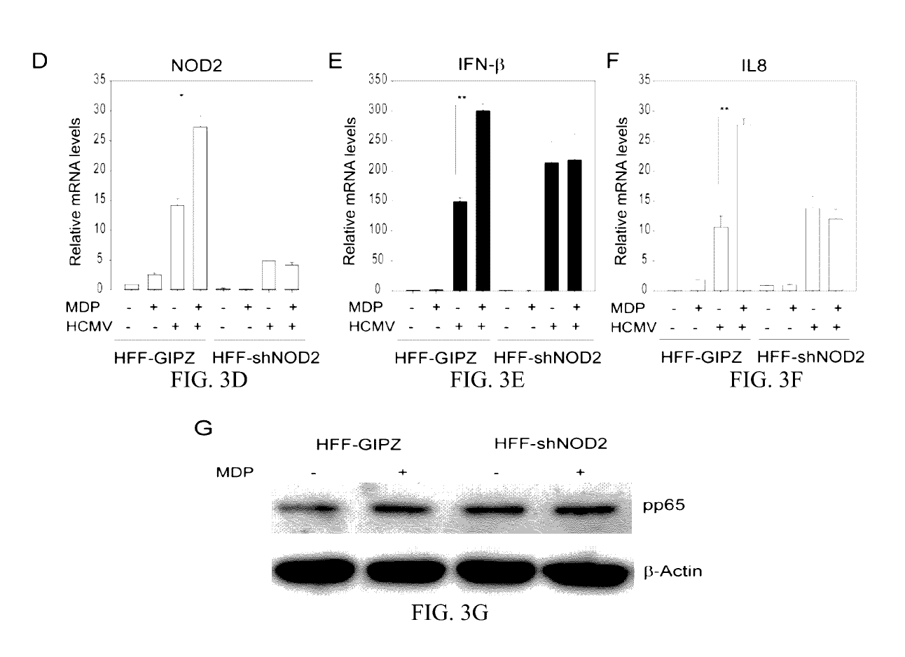

Since MDP directly binds to and activates NOD2, we investigated whether induction of IFN-β expression by MDP in infected cells was NOD2-dependent. The effects of MDP on NOD2, IFN-β, and IL8 transcripts was tested in HCMV-infected NOD2-KD (HFF-shNOD2) and GIPZ control HFFs (FIG. 3A-C). Similar to non-transduced HFFs (in FIG. 2), MDP induced NOD2, IFN-β and IL8 mRNAs in HCMV-infected control HFF-GIPZ cells. There was no such induction in HFF-shNOD2 irrespective of MOI used, confirming the requirement and specificity of NOD2 for MDP activities in HCMV-infected HFFs (FIGS. 3A&D). At MOI 3, NOD2 expression was significantly reduced in HFF-shNOD2 cells, but IFN-β and IL8 were induced in both control and NOD2 KD HFFs to similar degree by infection only or infection and MDP treatment (FIGS. 3E&F), suggesting that at high MOI NOD2-independent pathways could upregulate IFN-B and IL8 expression. The observed changes were not secondary to differences in infection efficiency between the cell lines used, since a Western blot for pp65 at 2 hpi showed similar virus entry in cells treated with MDP before infection (FIG. 3G). Taken together (FIGS. 1-3), MDP treatment at low MOI could enhance antiviral responses in a NOD2-dependent manner via activation of TBK1 and IRF3, resulting in decreased virus replication.

Prolonged Exposure to MDP Before Infection Inhibits HCMV Replication.

Since we previously reported that NOD2 induction by HCMV resulted in activation of downstream signaling and consequential inhibition of virus replication, and we now provide evidence that MDP suppresses virus replication, we tested whether ongoing MDP exposure prior to infection could generate an antiviral environment. HFFs were pretreated with MDP (10 μg/mL) for 72 h followed by infection with the Towne or TB40 strains of HCMV (100 PFU/well), and plaques were counted after 8 and 10 days, respectively. In MDP-pretreated cells plaque number was reduced by approximately 90% and 70% for Towne and TB40, respectively (FIGS. 4A&B). The effect of duration of MDP pretreatment on HCMV replication was next tested by pretreatment for 18 or 72 h followed by infection with the pp28 luciferase-recombinant HCMV-Towne (MOI 1). Luciferase activity measured in cell lysates at 96 hpi showed modest reduction of pp28 expression after 18 or 72 h MDP pretreatment (FIGS. 4C&D). Supernatants from non-pretreated and MDP-pretreated HCMV-infected cells were collected after 4 days (completion of first replication cycle) and used for infection of fresh HFFs for an additional 72 h (second cycle). Luciferase activity was reduced by approximately 45% and 75% in cells pretreated with MDP for 18 or 72 h, respectively (FIGS. 4 C&D). Supernatants from the first cycle infection were also used for virus titration in fresh HFFs and a plaque assay performed at 8 days post infection revealed a 6-fold reduction in virus titer in MDP-pretreated cells. (FIG. 4E). MDP pretreatment resulted in reduced expression of HCMV IE1/2, and pp65 (FIGS. 4F&G). The longer MDP pretreatment (72 h), the more significant reduction of IE2 and pp65 expression was observed in second cycle infection. Similar effects of MDP on HCMV replication were observed when a purified virus preparation was used (data not shown). NOD2 expression was induced in HCMV-infected cells at 96 hpi, and MDP pretreatment did not further increase NOD2 expression after infection (FIG. 4I). Compared to MOI 1 (FIG. 4D), at MOI 0.1, MDP pretreatment (10 μg/mL) reduced pp28 activity by 80% already during first cycle infection (FIG. 4I), and resulted in significant reduction of IE1/2 expression (FIG. 4J), suggesting that similar to the MOI dependency observed when MDP was added after infection, at lower MOI, MDP pretreatment was more efficient in virus inhibition. These effects were not secondary to differences in HCMV uptake, since a Western blot for pp65 at 2 hpi of MDP-pretreated or non-pretreated HFFs showed similar expression (FIG. 4K).

MDP Pretreatment Augments IFN-β Response in HCMV-Infected HFFs.

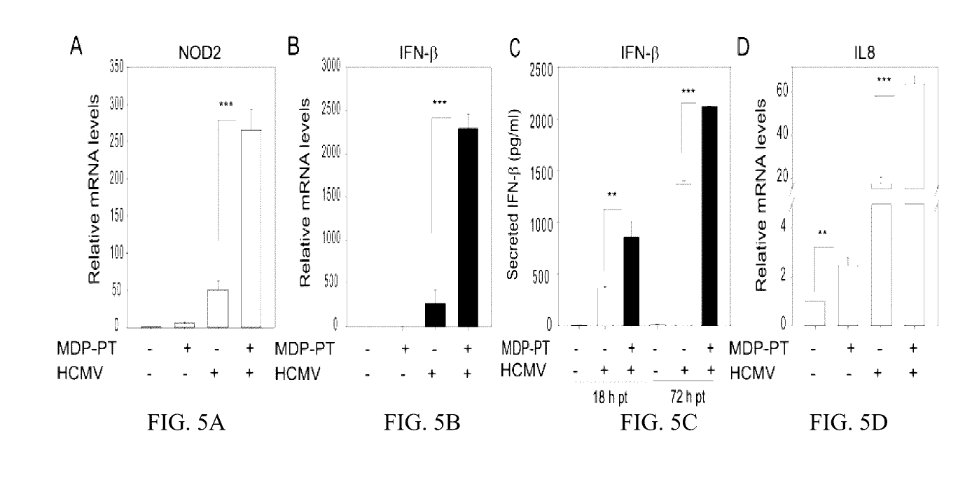

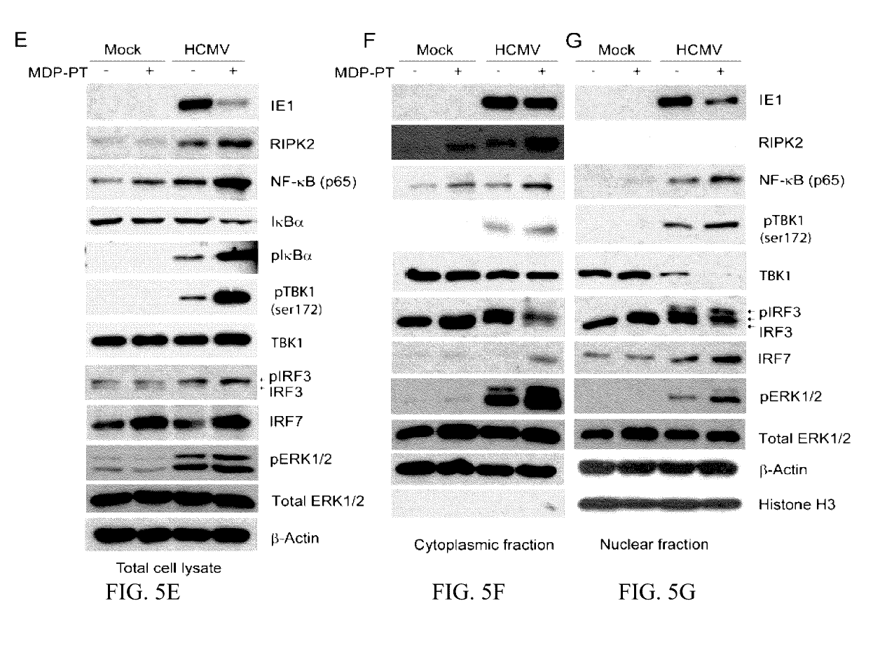

Since MDP pretreatment inhibited HCMV replication in HFFs (FIG. 4), and its addition following infection induced the phosphorylation of TBK, IRF3 and IFN-β expression (FIG. 2B, D, E), we hypothesized that the antiviral effects of MDP pretreatment were mediated through IFN-β. HFFs were pretreated with MDP (10 μg/mL) for 72 h followed by infection with HCMV Towne (MOI 1) for 24 h. NOD2, IFN-β and IL8 transcripts were quantified by qRT-PCR. Infection in MDP-pretreated cells induced NOD2 mRNA significantly more than infection only (265 vs 50-fold, FIG. 5A). A parallel experiment in non-infected cells (72 h MDP treatment followed by washing with PBS and incubation of the cells in 4% FBS containing medium for another 24 h) resulted in only 3-fold induction of NOD2 mRNA (FIG. 5A), suggesting the effects of MDP were augmented in infected cells. Similarly, infection in MDP-pretreated cells resulted in significant induction of IFN-β mRNA (FIG. 5B), and a parallel experiment in non-infected HFFs did not result in IFN-β induction. IFN-β levels were measured in supernatants collected from MDP-pretreated cells (for 18 or 72 h) and infected at MOI of 1 for 24 h by IFN-β-specific ELISA assay. Secreted IFN-β increased with the duration of MDP-pretreatment (FIG. 5C, p<0.01). MDP-pretreatment modestly induced IL8 expression, but there was no difference in induction between non-infected or HCMV-infected cells (FIG. 5D). These results demonstrate that exposure to MDP prior to HCMV infection significantly induces IFN-β mRNA upon infection as well as extracellular secreted IFN-β. The increased IFN-β response in MDP-pretreated HFFs may create an unfavorable environment for HCMV replication.

MDP Pretreatment Induces the Classical (NF-KB) and Alternative Pathways (TBK1-IRF3/7, pERK) Downstream of NOD2 in HCMV-Infected HFFs.

To understand the enhanced IFN-β responses in MDP-pretreated cells, the expression of proteins downstream of NOD2 and their cytoplasmic/nuclear localization was determined at 24 hpi. HCMV induced RIPK2 and NF-KB expression by approximately 6- and 4-fold, respectively (FIG. 5E, lanes 1&3), and a further increase was observed with MDP pretreatment (FIG. 5E lanes 3&4). NF-KB expression is regulated by inhibitory IκB proteins, which are regulated by upstream IκB kinases (IKKs). Phosphorylation of IκB proteins results in their degradation and release of the NF-KB complex. In MDP pretreated-HCMV-infected cells IκBα expression was decreased, accompanied by significant increase in phosphor-IκBα (FIG. 5E). The increase in RIPK2 expression was observed in the cytoplasmic fraction with no evidence for nuclear relocalization. MDP pretreatment increased NF-KB relocalization into the nuclear fraction of infected HFFs (FIGS. 5F&G). TBK1 phosphorylation was significantly induced in MDP-pretreated-infected cells (FIG. 5E) and pIRF3 was also increased. In the nuclear fraction total TBK1 expression was reduced but its phosphorylated form increased (FIG. 5G). Activated IRF3 (pIRF3) was increased in the cytoplasmic and nuclear fractions of infected HFFs. MDP-pretreatment followed by infection did not further increase pIRF3 and total IRF3 was decreased in the cytoplasmic fraction (FIG. 5F). However, a hyperphosphorylated form of IRF3 remained elevated in the nuclear fraction in infected MDP-pretreated HFFs.

IRF7 expression was increased in total cell lysates after MDP pretreatment of non-infected and HCMV-infected cells (FIG. 5E). In the cytoplasmic fraction IRF7 was only detected in MDP-pretreated infected HFFs. Infection mildly increased nuclear IRF7 expression and a further increase was observed in MDP-pretreated HCMV-infected HFFs, suggesting that MDP triggered IRF7 localization and possible activation in the nuclear fraction of infected cells (FIG. 5G). Levels of pERK1/2 were also increased in MDP-pretreated HCMV-infected cells compared to infection only. These changes reflect an increase in ERK phosphorylation, since the expression of total ERK was unchanged. The expression of pERK2 (p42 MAPK) was increased in MDP-pretreated infected HFFs in total cell lysates, cytoplasmic and nuclear fractions, compared to infection only (FIGS. 5E, F&G) whereas pERK1 (p44 MAPK) increased in total cell lysates, and in the cytoplasmic fraction (FIGS. 5E&F). As expected, at 24 hpi HCMV-IE1 expression was reduced in MDP-pretreated cells, compared to non-pretreated cells (FIG. 5E-G).

The Anti-HCMV Activities of MDP Require IFN-β and are Significantly Attenuated in IFN-β KD HFFs.

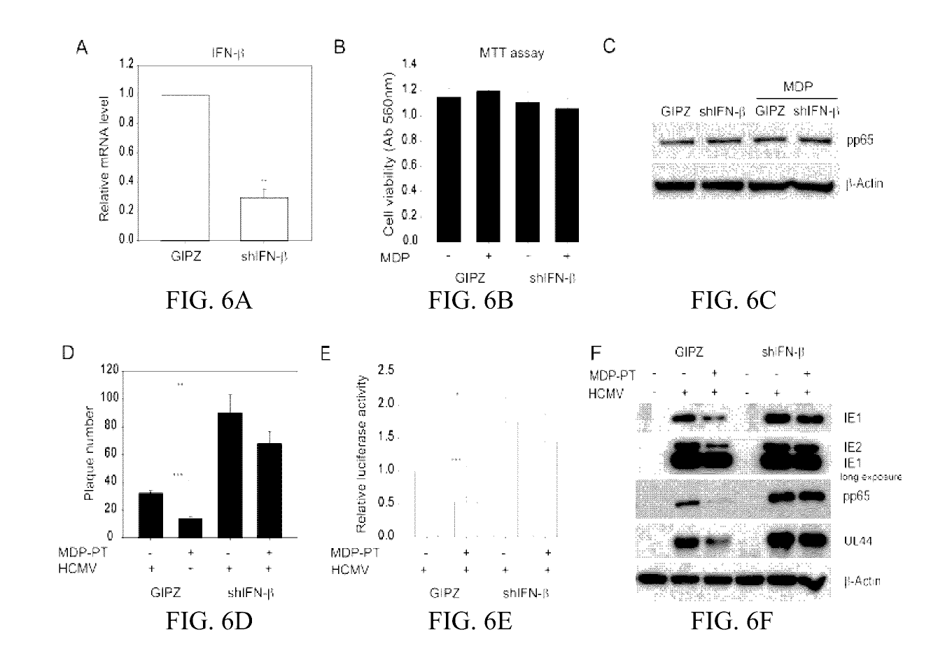

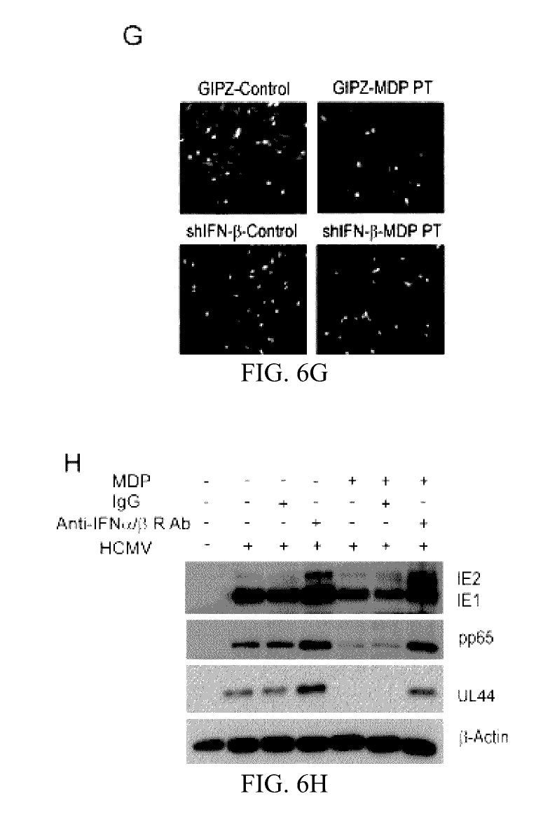

Since MDP pretreatment inhibited HCMV replication, and higher IFN-β response was elicited in MDP-pretreated infected HFFs, we hypothesized that the augmented NOD2-dependent IFN-β response could be critical for suppressing HCMV replication. To address this, HFFs stably expressing shRNAs against the IFN-β gene were generated using lentiviral vectors. Five clones expressing different lentiviral shRNAs were generated and the clone resulting in the highest KD of IFN-β expression was selected for additional experiments (FIG. 6A). Equal number of control (GIPZ) and IFN-β-shRNA expressing cells (shIFN-β) were mock treated or pretreated with MDP (10 μg/mL) for 72 h and after 3 days cell viability was determined by MTT assay. No toxicity or difference in cell growth was observed in MDP-pretreated control GIPZ or shIFN-β KD cells (FIG. 6B). Virus entry, determined by a Western blot for pp65 at 2 hpi, was similar among the different cell lines, with or without MDP pretreatment (FIG. 6C). To investigate the role of IFN-β in MDP-induced restriction of HCMV replication, a plaque assay was performed in control GIPZ and shIFN-β cells pretreated with MDP for 72 h, followed by infection with HCMV Towne (100 PFU/well). The number of plaques, counted at day 8 post infection, was significantly decreased in MDP-pretreated control GIPZ cells while in shIFN-β cells plaque number increased. In shIFN-β cells treatment with MDP failed to restrict virus replication and the plaque number was similar between control/untreated vs MDP pretreated cells (FIG. 6D). Similarly, pretreatment with MDP for 72 h followed by infection with the pp28 luciferase-recombinant Towne strain for 72 h (MOI 1) revealed decreased pp28-luciferase activity in control GIPZ cells, while in MDP-pretreated shIFN-β cells, pp28-luciferase activity was enhanced (FIG. 6E). MDP pretreatment of control GIPZ cells resulted in reduced IE1/2 expression as well as significant reduction of UL44 and pp65 expression (FIG. 6F). The expression of UL44, pp65 and IE1/2 in shIFN-β cells was increased compared to control (GIPZ) cells, and MDP pretreatment in these cells had no effect on the expression of viral proteins, demonstrating that the antiviral effects of MDP were mediated through IFN-β (FIG. 6F). An immunofluorescence staining of nuclear IE1/2 in GIPZ- and shIFN-β-HFFs, pretreated with MDP for 72 h, and infected for 24 h with a clinical isolate of HCMV showed reduced IE1/2 expression in GIPZ cells, but not in the shIFN-β cells (FIG. 6G). An IFN-β receptor blocking antibody added to infected cells also attenuated the antiviral activity of MDP (FIG. 6H). These results demonstrate that IFN-β plays an important role in the anti-HCMV activities of MDP.

Discussion

NOD2, a PRR for bacterial pathogens and a susceptibility marker for Crohn's disease, was recently reported to recognize RNA viruses, respiratory syncytial virus (RSV), influenza, parainfluenza, and we have reported on HCMV recognition by NOD2. Induction of NOD2 by HCMV triggers an antiviral cytokine response and results in reduced virus replication. In cells overexpressing the NOD2 mutant (3020C), HCMV replication is not inhibited and IFN-β is not induced. These results suggest that NOD2 activation by MDP may restrict HCMV replication, and in a larger scope, a role for bacterial environment in controlling HCMV replication.

We report here on the anti-HCMV activities of MDP, when used either after or before infection. In both cases IFN-β induction downstream of NOD2-RIPK2-TBK1 was a predominant pathway for virus inhibition. MDP treatment at low MOI (0.1) induced the IFN-β pathway downstream of NOD2, evident by increased phosphorylation of TBK1 and IRF3. This effect was MOI-dependent and observed only at low MOI. There was also induction and nuclear translocation of NF-KB by MDP treatment at low MOI. Neither pTBK1, pIRF3, nor NF-KB were induced in MDP-treated cells after infection at MOI 1, suggesting that NOD2-dowenstream signaling cannot overcome a high titer infection. Restriction of HCMV replication by IF116 was also reported to be MOI dependent, although it was independent of IFN-β. MDP activity in HCMV-infected cells was mediated through NOD2, since in NOD2 KD cells IFN-β was not induced. The anti-HCMV activity of MDP was dose-dependent and occurred at an early stage of HCMV replication, between 8 h and 48 hpi.

The inhibition of HCMV replication with MDP pretreatment was also more efficient at low MOI, was time-dependent and increased with longer exposure. In MDP-pretreated HFFs, pTBK1, a hyperphosphorylated form of IRF3 and IRF7 were increased in the nuclear fraction, suggesting that TBK1 activation downstream of NOD2 induced antiviral response through IRF3/7. Activated (phosphorylated) IRF7 was reported to form heterodimers with IRF3 and promote increased expression of IFN-β. IRF7 can be activated through pathways mediated by TLR3, -7 and -9, RIG-I and likely DAI and IF116, as well as by TLR2-mediated signaling pathway. Infection with Helicobacter pylori induced RIPK2 by NOD1, activated IκB kinase ε (IKKε) and IRF7, followed by synthesis of type I IFN and signaling of the latter through IFN-stimulated gene factor 3 (ISGF3), suggesting that IRF7 might be activated through NLRs. Using IFN-β KD HFFs we show that the anti-HCMV activities of MDP are mediated predominantly through IFN-β. Cellular interferon-responsive gene expression (ISG15, ISG54) is induced upon infection of HFFs with HCMV. Our results using IFN-β receptor blocking antibody suggest that MDP may stimulate the expression of certain interferon-stimulated genes (ISGs) via induction of IFN-β expression. Some of these ISGs are likely required for the anti-HCMV activity of MDP via NOD2.

The role of NOD2 in viral recognition is just beginning to be uncovered and most information is available from studies of RNA viruses. Infection with RSV activated NOD2-mitochondrial antiviral signaling protein (MAVS) and the IFN pathway. RSV infection induced IFN-β, resulting in upregulation of NOD2. Subsequent activation by MDP induced higher proinflammatory cytokine response, suggesting that mucosal colonization with bacterial components might enhance proinflammatory cytokine responses and lead to more severe RSV disease in young children. Similarly, infection of murine macrophages with murine norovirus-1 (MNV1) induced NOD1 and NOD2 in an IFN-β-dependent manner, and subsequent bacterial infection enhanced activation of NOD1/2, mediated by type I IFNs. Crosstalk between type I IFNs and NOD1/NOD2 signaling has been suggested to promote bacterial recognition, resulting in harmful effects in virally-infected host. These studies differ from ours in that MDP was only used after (not before) virus infection and we investigated the effects of MDP both prior to and after HCMV infection. In addition, activation of innate/adaptive immune responses against these RNA viruses are expected to differ from those activated by HCMV, since the RNA viruses will eventually be cleared while HCMV must employ mechanisms to counteract NOD2 recognition, achieve productive replication and latency. Some of these mechanisms may involve interference with or modulation of signaling downstream of NOD2.

Recent studies are beginning to identify a role for the gut virome in disease processes. Using stool samples from patients with inflammatory bowel disease, changes in the virome were suggested to contribute to intestinal inflammation and bacterial imbalance. Herpesvirus latency was suggested to confer symbiotic protection from bacterial infection. In a mouse model of latent mouse CMV infection, resistance to Listeria monocytogenes and Yersinia pestis was observed, suggesting that latency may upregulate the basal activation state of innate immunity against subsequent bacterial infection. At this time there are no data on the role of NOD2 in mouse CMV infection; based on our in vitro data it is possible that bacterial decolonization may result in a more conducive environment for HCMV replication.

In summary, our study presents the unique activities of bacterial MDP on HCMV suppression via NOD2 activation either at the time of infection or in the setting of persistent NOD2 activation. The latter may represent changes in bacterial colonization which commonly occurs in vivo. Intestinal expression of NOD2 has been suggested to be regulated by the microbiota, since germfree mice had lower NOD2 expression that was restored upon colonization with commensal bacteria. Our data may support a model for bacteria-HCMV cross talk providing a protective antiviral environment.

### Example 2

### Inhibition of Mouse CMV (MCMV) Replication with iE-DAP

Animal work was carried out in strict accordance with the recommendations in the Guide for the Care and Use of Laboratory Animals of the National Institutes of Health. The animal protocol (number MO13M296) was approved by the Institutional Animal Care and Use Committee (IACUC) of Johns Hopkins University. For infection experiments 3-4 week old BALB/c mice were purchased from Harlan Laboratories (Indianapolis, Ind.). After 2-3 days of adaptation to the housing environment, mice were pretreated with iE-DAP (two daily doses of 500 μg each) (Cardenas et al., 187(2) J. IMMUNOL. 980-86 (2011)) or saline intraperitoneally. Blood was collected 4 h after the second dose of iE-DAP for measurement of RANTES by ELISA (R&D Biosystems, Minneapolis, Minn.). Mice were infected with 10⁶ PFU tissue culture derived MCMV (Smith strain, ATCC, VR-1399) and scarified after 14 days. Intracardiac blood was collected prior to sacrifice for gB real-time PCR assay (Vliegen et al., 98(1) VIRUS RES. 17-25 (2003)). Salivary glands, liver and spleen were harvested and stored at −80° C. Organs were homogenized in DMEM at a final concentration of 100 mg/mL Two million mouse embryonic fibroblast (MEFs) were seeded into 24-well plates. From each sample, 2.5% of the salivary gland or 10% of liver/spleen homogenate was used for infection of MEFs in triplicates. Plaques were counted after three days.

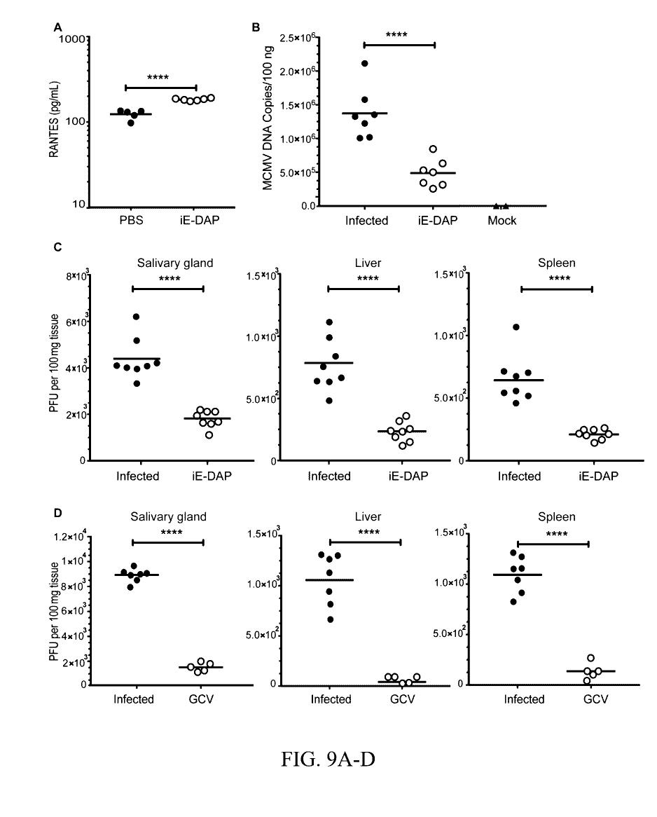

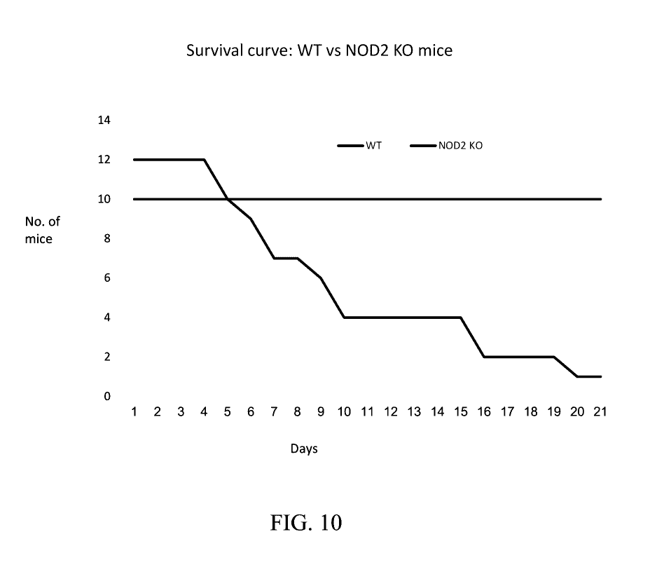

In vivo NOD1-dependent anti-MCMV activity: BALB/c mice (3-4 week) were pretreated with iE-DAP (Invivogen, San Diego, Calif.), 500 μg once daily for two days, followed by infection with MCMV at 10⁶ PFU/mice. iE-DAP activity was confirmed by induction of RANTES in serum samples collected 4 h after administration of the second dose (FIG. 9A). At 14 days' post infection mice were scarified, intracardiac blood was collected and tissue homogenates were prepared for plaque assays. In iE-DAP pretreated mice real-time PCR for gB (FIG. 9B) and plaque numbers in salivary glands, liver and spleen (FIG. 9C) were significantly reduced as compared to infected only mice (p<0.0001). Ganciclovir (GCV), used as a direct anti-viral agent inhibited MCMV, as expected (FIG. 9D).

We claim:

1. An immunogenic composition comprising (a) a live, attenuated or replication-defective cytomegalovirus composition and (b) a NOD1 activator and/or a NOD2 activator.

2. The composition of claim 1, wherein the NOD2 activator is muramyl dipeptide (MDP) or a derivative thereof.

3. The composition of claim 1, wherein the NOD1 activator is L-Ala-γ-D-Glu-meso-diamino-pimelic acid (tri-DAP) or a derivative thereof.

4. An immunogenic composition comprising (a) a CMV immunogen composition and (b) a NOD1 activator and/or a NOD2 activator.

5. The composition of claim 4, wherein the NOD2 activator is MDP or a derivative thereof.

6. The composition of claim 4, wherein the NOD1 activator is tri-DAP or a derivative thereof.

7. A pharmaceutical composition comprising a CMV adjuvant, wherein the CMV adjuvant comprises MDP and/or tri-DAP or derivatives thereof.
